(19) United States
(12) Patent Application Publication
(10) Pub. No.: US 2016/0038722 A1
(43) Pub. Date: Feb. 11, 2016

(54) CUTTING EDGE CUTTING CATHETER

(71) Applicant: Cook Medical Technologies LLC, Bloomington, IN (US)

(72) Inventors: Cameron McLain, Columbus, OH (US); David Hagan, Louisville, KY (US); John Tyler Strader, Elizabethtown, KY (US); Marie Hopkins, Cleveland Heights, OH (US)

(21) Appl. No.: 14/818,853

(22) Filed: Aug. 5, 2015

Related U.S. Application Data
(60) Provisional application No. 62/033,735, filed on Aug. 6, 2014.

(51) Int. Cl. A61M 25/10

(52) U.S. Cl. CPC A61M 25/104; A61B 2017/320791

(57) ABSTRACT

The present disclosure provides a cutting edge cutting catheter and a method for treatment of peripheral chronic total occlusion (“CTO”) in a body vessel. The cutting edge cutting catheter has an arcuate cutting edge slidably disposed within an elongate member or catheter. A tension mechanism connects to the cutting edge and applies a tension to move the cutting edge between a bent state and a straight state. An expandable balloon surrounds the elongate member to dilate the occluded body vessel and ablate the occlusion.

### CROSS-REFERENCE TO RELATED APPLICATIONS

This application claims priority to U.S. Provisional Patent Application Ser. No. 62/033,735, filed Aug. 6, 2014, entitled “CUTTING EDGE CUTTING CATHETER,” the entire contents of which are hereby incorporated by reference.

### BACKGROUND

1. Technical Field

The present disclosure relates to medical devices. More particularly, the disclosure relates to a cutting edge cutting catheter to treat peripheral chronic total occlusion (“CTO”).

2. Background Information

Peripheral chronic total occlusion (“CTO”) is when plaque accumulates in a blood or body vessel so no or little blood can flow through the vessel. This is often a very painful and dangerous condition, as it may cause ischemia in the extremities requiring invasive treatment.

Treatment for CTO is to clear the occlusion from the blood vessel. This may involve exercise, pharmacological methods, invasive surgery, or endovascular and interventional methods. However, traditional treatment methods may not be successful due to the nature of the occlusion. For example, a cholesterol crystal cap may form at the ends of the occlusion. This cap forms due to macrophage accumulation in an inflammatory response and an increase in retained Low Density Lipoprotein (“LDL”). This cholesterol crystal cap creates a hard surface that is difficult for the physician to maneuver through in order to clear the vessel.

One minimally invasive treatment method is the STAR (subintimal tracking and re-entry) method. This method involves using medical tools, such as a catheter, to maneuver from the true lumen of the blood vessel, through the intimal layer, into the subintimal space. Once the user or physician passes the occlusion by way of the subintimal space, the user maneuvers back from the subintimal space into the true lumen. At this point, the physician can access the occlusion.

Using the STAR method requires making fine maneuvers in the blood vessel with a high degree of precision at a position deep within the body. Any unintended cuts may create avoidable issues. Thus, there is a need for an improved device that can make these fine maneuvers to avoid unintended cuts.

### BRIEF SUMMARY

The present disclosure generally provides a cutting edge cutting catheter or medical device suitable for peripheral CTO treatment. The present disclosure also generally provides a method for treating an occlusion within the blood vessel.

The device comprises an elongate member, a cutting edge, an extending member, a tension mechanism, and an expandable balloon. The elongate member may have a proximal end extending distally to a distal end, a circumference, and a plurality of lumens formed therethrough. The plurality of lumens comprising a cutting lumen, a balloon inflation lumen, and a wire guide lumen. The device further comprises a cutting edge slidably disposed within the cutting lumen and having an arcuate cross-section. The cutting edge arcuately extends circumferentially up to half of the circumference, and comprises a first end extending distally to a second end, the second end being adjacent to the distal end, the cutting lumen being formed complementary to the cutting edge.

As one advantage, the arcuate shape of the cutting edge may allow the device to contain a larger cutting edge relative to the elongate member, while the elongate member may be smaller. This allows the elongate member to maneuver within the vasculature more precisely, reducing unintended trauma.

The cutting edge may comprise a flexible material. This may be a shape memory material, such as Nitinol. The cutting edge may be controlled by an extending member being attached to the cutting edge adjacent to the proximal end. The extending member is operable to slidably move the cutting edge relative to the cutting lumen, defining an extended state and a retracted state of the cutting edge. In one embodiment, the cutting edge extends from the proximal end to the distal end.

The tension mechanism may be connected to the proximal end and extend to the cutting edge. The tension mechanism, moveable in a predetermined direction, applies a tension to the cutting edge to bend the cutting edge, defining a straight state and a bent state of the cutting edge. Through the tension mechanism and the extending member, the physician may have increased control over the cutting edge to maneuver it into and out of the subintimal space. As such, the elongate member may form a track closer to the proximal end than the distal end. The extending member may include a button slidably received in the track. The retracted state allows the physician to avoid undesirable cuts to the vessel wall.

The expandable balloon may be disposed circumferentially about the elongate member and in fluid communication with the balloon inflation lumen wherein the expandable balloon moves from a collapsed state to an expanded state to treat or ablate the occlusion. As one advantage, because the device contains the expandable balloon, this may allow the physician to have the expandable balloon and the cutting edge within the same device, to cut around and dilate the occlusion with the same device.

The elongate member comprises a plurality of side ports disposed adjacent to the distal end to provide fluid communication between the balloon inflation lumen and the expandable balloon. The expandable balloon is disposed proximal to the distal end. In one embodiment, the proximal end comprises a handle, and the tension mechanism comprises a tension core. The tension core is connected to the handle and extends distally to the cutting edge to move the cutting edge between the straight and bent states. In one embodiment, the tension core comprises a wire.

The handle being integrally formed with the elongate member at the proximal end to rotate the device. For example, rotating the handle rotates the distal end. The handle further comprises a tension lever being movable (e.g. depressible and releasable) to move the cutting edge between the straight and bent states. In another embodiment, a push member is connected to the proximal end and extends distally to the first end, the first end being disposed distal from the proximal end and extending to the distal end.

In one form, the device further comprises an L-shaped radiopaque marker at the distal end, the radiopaque marker being disposed adjacent to the cutting lumen for fluoroscopy.

The disclosure provides a method for treating an occlusion in a body vessel having a true lumen and a subintimal space, the occlusion having a first side and a second side. The method may include first, positioning a wire guide and a medical device in the true lumen adjacent to the first side, the medical device as described above; second, advancing the distal end of the elongate member from the true lumen into the subintimal space of the body vessel having the cutting edge in the extended state; third, advancing the distal end from the subintimal space into the true lumen of the body vessel and adjacent to the second side having the cutting edge in the extended state; fourth, aligning the expandable balloon with the occlusion, the expandable balloon being maintained within the subintimal space, and; fifth, ablating the occlusion.

Each step of advancing the distal end may comprise first, rotating the device about the circumference and second, applying the tension on the cutting edge to move the cutting edge from the straight state to the bent state or the bent state to the straight state. After each step of advancing the distal end, the method may comprise first, retracting the cutting edge to the retracted state and second, moving the distal end through the body vessel after the step of retracting. The step of ablating the occlusion further comprises first, inflating and second, deflating the expandable balloon.

### BRIEF DESCRIPTION OF THE DRAWINGS

FIG. 1 is an partial, environmental side view of a medical device for treating an occlusion in a body vessel in accordance with one embodiment of the present invention;

FIG. 2 is a partial cross-sectional view of a distal portion of the device in FIG. 1 taken along line 2-2;

FIG. 3 is a partial side view of the distal portion in FIG. 1;

FIG. 4 is an end view of the distal portion in FIG. 1;

FIGS. 5A and 5B are partial cross-sectional views of a retracted state and an extended state, respectively, of a cutting edge in FIG. 1;

FIG. 6 is a partial cross-sectional view of a bent state of the cutting edge in FIG. 1;

FIG. 7 is a partial cross-sectional view of a straight state of the cutting edge in FIG. 1;

FIG. 8 is a partial cross-sectional view of an occluded blood vessel;

FIG. 9 depicts the steps of one method of treating the occluded blood vessel with the medical device of FIG. 1 in accordance with one example of the present invention;

FIG. 10 is a flow diagram of the method in FIG. 9; and

FIGS. 11A and 11B show a delivery assembly for introducing the medical device of FIG. 1 into the vasculature.

### DETAILED DESCRIPTION

The present disclosure generally provides a cutting edge cutting catheter suitable for treating CTO. The present disclosure also provides one example of a method for treating CTO. The disclosure provides embodiments of the medical device and the process, and the cited figures illustrate these embodiments. The accompanying figures are provided for general understanding of the structure of various embodiments. However, this disclosure may be embodied in many different forms. These figures should not be construed as limiting and they are not necessarily to scale.

All publications, patent applications, patents, and other references mentioned herein are incorporated by reference in their entirety. Unless otherwise defined, all technical and scientific terms used herein have the same meaning as commonly understood by one of ordinary skill in the art to which this disclosure pertains. In case of conflict, the present document and definitions will control.

“Adjacent” referred to herein is nearby, near to, or in close proximity with.

“Ablate” referred to herein is to reduce or crush a vessel occlusion.

“Adventitial layer” referred to herein is the outer surface of the blood vessel wall, farthest away from healthy blood flow.

To “bend” the cutting edge means to move the cutting edge into or toward its bent state or straight state.

“Intimal layer” referred to herein is the inner surface of the blood vessel wall, closest to healthy blood flow.

The terms “proximal” and “distal” and derivatives thereof will be understood in the frame of reference of a medical physician using the medical device; thus, proximal refers to locations closer to the physician and distal refers to locations further away from the physician (e.g. deeper in the patients vasculature).

“Subintimal space” or “subintimal layer” is area within the intimal and adventitial layers of the blood vessel walls.

“True lumen” referred to herein is the normal or healthy pathway for blood flow within the vasculature.

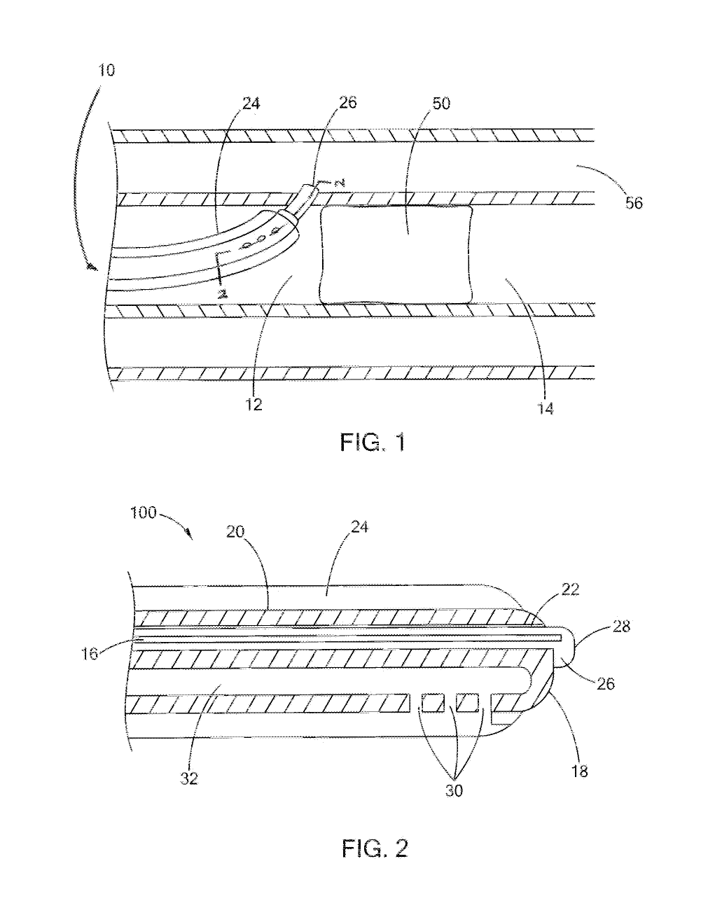

FIG. 1 illustrates the device 10 within the body vessel. FIG. 1 depicts the device 10 entering the subintimal space 56 on the first side 12 of the occlusion 50. The device 10 may bypass the occlusion 50, and re-enter on the second side 14 of the occlusion. The size and features of the vessel in FIG. 1 are exaggerated here for clarity, and may not be to scale.

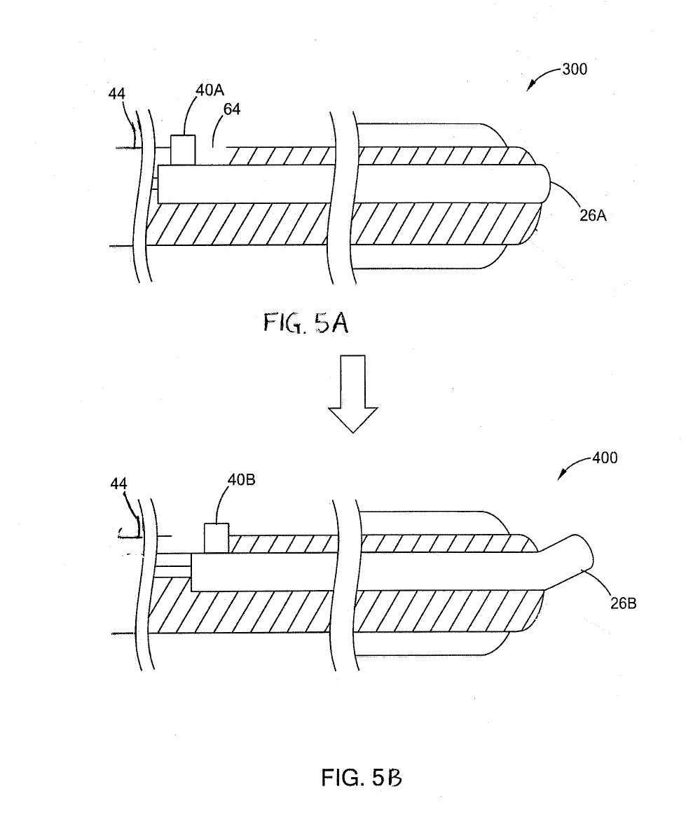

FIG. 2 illustrates a partial cross-section 100 of the device of FIG. 1 along line 2-2. The device comprises an elongate member 20 having a proximal end (discussed with FIG. 5A (44)) extending distally to a distal end 18. The elongate member has a circumference and a plurality of lumens formed therethrough. The elongate member 20 may be manufactured by polymer extrusion. In one form, the length of the elongate member 20 may be long enough to reach the occlusion. While only a portion of the device is shown in FIG. 2, it is understood that the total length of the elongate member may be between about 50 centimeters and about 80 centimeters.

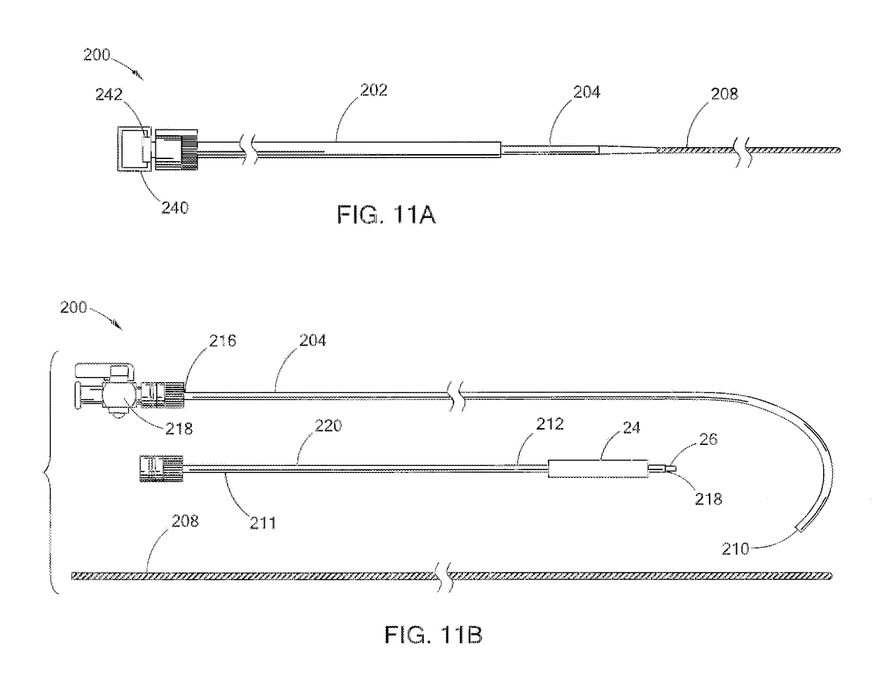

The elongate member 20 may comprise at least three lumens: the cutting lumen 22, the balloon inflation lumen 32, and the wire guide lumen (discussed with FIG. 4 (38)). The wire guide lumen may house a wire guide (discussed with FIG. 11B (208)) to position the device adjacent to the body vessel. In one example, the wire guide is inserted first. In this example, the wire guide guides the elongate member or catheter as it slides along the wire guide by way of the wire guide lumen. This process may orient the elongate member adjacent to the first side occlusion.

In one embodiment, the cutting lumen 22 is formed in the elongate member opposite from the balloon inflation lumen 32. The wire guide lumen may be formed in the center of the elongate member 20. It is understood that the lumens may be formed in different locations of the elongate member 20 without falling beyond the scope and spirit of the present invention.

An expandable balloon 24 may be disposed circumferentially about or around the elongate member 20. In this embodiment, the expandable balloon 24 is in fluid communication with the balloon inflation lumen 32 through a plurality of side ports 30. In one aspect, three side ports are positioned proximal to the distal end 18 of the elongate member 20 to provide fluid communication between the balloon inflation lumen and the expandable balloon. It is understood that the number and position of the side ports may vary. For example, the side ports may be located at any position along the expandable balloon. In one aspect, the expandable balloon 24 is located several centimeters proximal to the distal end 18 of the elongate member 20. The expandable balloon may move from a collapsed state to an expanded state to treat the occlusion 50.

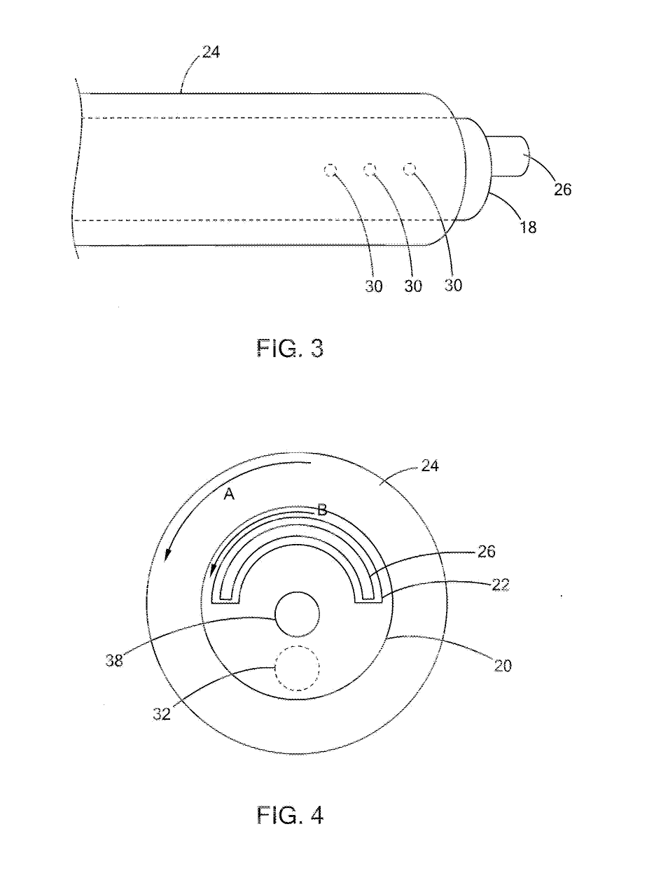

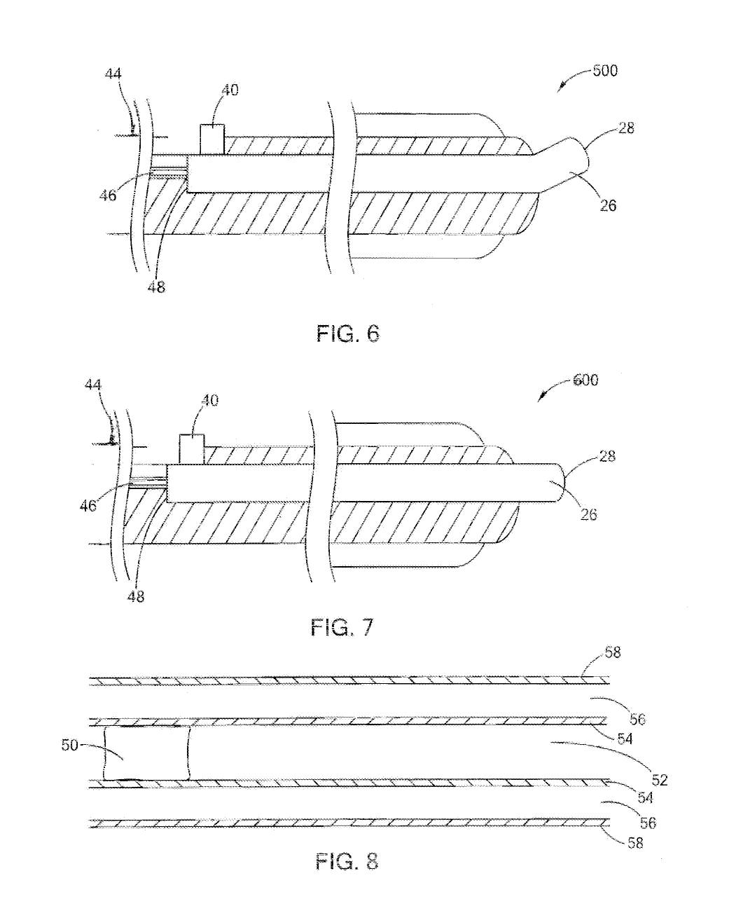

A cutting edge 26 or blade may be slidably disposed within the cutting lumen 22 and may be arcuate. The cutting edge has an arcuate cross-section, arcuately extends circumferentially up to half of the circumference. In one form, the cutting edge has a first end (discussed with FIG. 6 (48)) extending distally to a second end 28. The second end 28 may be located at the distal end 18 of the elongate member in the retracted position. When the second end 28 is in the extended position, it protrudes or extends beyond distal end 18. FIGS. 2-3 show the cutting edge in the extended state, extending beyond the distal end 18 of the elongate member. The cutting edge 26 also has a retracted state, discussed below.

The cutting edge 26 may be manufactured by laser cutting. Further, the cutting edge 26 may be formed from any suitable material for CTO. Such material may be flexible. Further, such material may be thin to penetrate the intimal layer 54. In one embodiment, the cutting edge 26 is blunt and thin that allows it to penetrate into the subintimal space. The cutting edge 26 does not need to be sharp. The material to form the cutting edge 26 will be a thin, flexible material, such as stainless steel. In one form, the material will be a shape memory material (e.g. Nitinol).

The device also comprises a tension mechanism. The tension mechanism contains a tension core to bend the cutting edge as part of the tension mechanism. The tension core may be wire 16 which bends and straightens the cutting edge 26. Wire 16 may be connected to the proximal end and extend distally to the cutting edge. When moved in a predetermined direction, the tension mechanism applies a tension to the cutting edge to bend the cutting edge, defining a straight state and a bent state of the cutting edge. If the tension core is mechanical, it may be attached to the cutting edge through any method known in the art, such as welding, soldering, gluing or chemical bonding. A skilled artisan will understand that the tension core could also be any means known in the art to bend the cutting edge, such as a mechanical, electric, magnetic, or pneumatic mechanism.

FIG. 3 illustrates the expandable balloon 24. In this view, expandable balloon 24 surrounds the elongate member, and is located proximal to distal end 18. Expandable balloon 24 is in fluid communication with the balloon inflation lumen through side ports 30.

FIG. 4 illustrates an end view of the device. Also in this aspect, the expandable balloon 24 is disposed circumferentially about elongate member 20. The expandable balloon moves between a collapsed state and an expanded state. When the expandable balloon is collapsed, the balloon circumference A is about the same or equal to the elongate member circumference B. “About” may mean within 20%, within 10%, or within 5%. In the expanded state, the balloon circumference A is greater than the elongate member circumference B.

FIG. 4 also shows the shape of the cutting edge 26. In one form, the cutting edge is arcuate, shown concave down on the top side of the elongate member 20. The physician can alternatively rotate the elongate member such that the cutting edge 26 is concave up and on the opposite side of the elongate member from its position in FIG. 4 using the handle at the proximal end. In one aspect, the cutting edge 26 extends around the elongate member circumference B up to and including half of the circumference B. The cutting lumen 22 is formed complementary to accommodate the arcuate cutting edge 26. Wire guide lumen 38 is shown in the center of the elongate member 20. The balloon inflation lumen 32 is shown in dotted lines as it is not formed through to the distal end of the elongated member 20. The balloon inflation lumen 32 may be positioned on the opposite side of the elongate member from the cutting lumen 22.

A physician may tell which way the cutting edge faces with an L-shaped, radiopaque marker through fluoroscopy. Such a marker may be located on the side of the elongate member 20 next to or adjacent to the cutting edge 26 at the distal end. In one embodiment, when the physician views an L-shape, the cutting edge 26 is concave down. When the physician views a backwards L-shape, the cutting edge 26 is concave up.

FIGS. 5A and 5B depict cross-sectional views of a retracted and extended state of the cutting edge. The device may contain an extending member 40 attached to the cutting edge adjacent to the proximal end 44. Extending member 40 is operable to slidably move the cutting edge relative to the cutting lumen between its retracted position 300 and its extended position 400. The extending member 40 is located adjacent to the proximal end 44 of the elongate member, as shown in FIGS. 5A-B. It moves between positions 40A and 40B to move from a retracted state 300 to an extended state 400. This sliding moves the cutting edge between retracted position 26A and extended position 26B, respectively. The extending member 40 may be a button and be disposed in track 64, and may only extend the cutting member as far as track 64 allows. Track 64 may be formed in the elongate member closer to the proximal end than the distal end. In one form, this provides a positive stop for the cutting edge. In the retracted position 26A, the cutting edge may be flush or even with the distal end even though a slight protrusion is shown in FIG. 5A.

When the physician makes a desired cut in the vasculature, the cutting edge 26 may be extended in extended state 400. When the physician desires to move the elongate member within the true lumen or within the subintimal space, the cutting edge 26 may be in retracted position 300 to avoid the risk of making an unintended cut.

FIGS. 6 and 7 depict cross-sectional views of a bent state 500 and a straight state 600 of the cutting edge. In addition to the retracted and extended states, the cutting edge also has a bent state 500 and a straight state 600. In one embodiment, the cutting edge 26 can be biased to form the bent state 500, and straighten or bend upon using the tension mechanism. In another embodiment, the cutting edge may be biased to be straight, and can bend upon applying the tension.

As shown in FIG. 6, the second end 28 of the cutting edge 26 is biased to bend. In one embodiment, when the cutting edge exits the elongate member, it may automatically assume a bent state 500. This may be accomplished based on the material used. For example, if the material is a shape memory material (e.g. Nitinol) it may be heat set or predisposed to bend upon exiting the constraints of the elongate member 20 and the cutting lumen 22. Alternatively, the second end 28 of the cutting edge 26 may be biased to be straight upon exiting the elongate member.

In either case, the cutting edge 26 may be further manipulated by a tension mechanism. In one embodiment the tension mechanism may be a wire (shown in FIG. 2 (16)), which is connected to the proximal end 44. The wire may be connected to a tension lever 242 at handle 240 (discussed with FIG. 11A) and extend distally to the cutting edge to move the cutting edge between the straight and bent states. The wire may be connected adjacent to the second end 28. The user may alternatively pull or push the tension lever 242 in the handle 240 and apply the tension through wire to the second end 28 of the cutting edge 26. Such tension may move the cutting edge between a bent state and a straight state. In one aspect, the wire may bend the second end in one direction. Alternatively, the wire may be attached on the opposite side of the cutting edge 26 to bend the second end 28 in another direction to straighten the cutting edge. The tension mechanism may bend the blade in a controlled manner to achieve precise manipulation of the cutting edge through the vasculature.

FIGS. 6 and 7 depict another feature of the cutting edge 26. In one aspect, the cutting edge 26 may extend from the proximal end 44 to the distal end, the entire length of the elongate member 20. In this case, the cutting edge may comprise plastic. Alternatively, the cutting edge 26 may extend only a portion of the length of the elongate member 20. In this case, the cutting edge 26 has the first end 48 connected to a push member 46. The push member 46 may be connected to the proximal end 44 and extend to the first end 48. The first end 48 may be disposed distal from the proximal end 44. The first end 48 may extend to the distal end.

In one aspect, the push member 46 is a round wire. In another aspect, the push member is a flat wire. A skilled artisan will understand that the push member may be any shape to connect to the cutting edge without falling beyond the scope and spirit of the disclosure. In any case, the cutting lumen is formed complementary to the push wire 46 adjacent to the proximal end and formed complementary to the cutting edge 26 adjacent to the distal end. The wire may be attached to the cutting edge through any method known in the art, such as welding, soldering, gluing or chemical bonding.

FIGS. 8 through 10 depict a method of treating the occlusion. FIG. 8 shows a cross-sectional view of a blood vessel. The blood vessel contains true lumen 52 situated or formed within the vessel wall. The vessel wall is composed of the adventitial layer 58 and the intimal layer 54. The intimal layer 54 surrounds the true lumen 52. The subintimal space 56 is formed within the intimal and adventitial layers. In this figure, occlusion 50 completely blocks the body vessel.

Although not explicitly depicted, the body vessel has a medial layer between the adventitial layer 58 and the intimal layer 54. When the device enters the subintimal space 56, it may also penetrate into or through the medial layer.

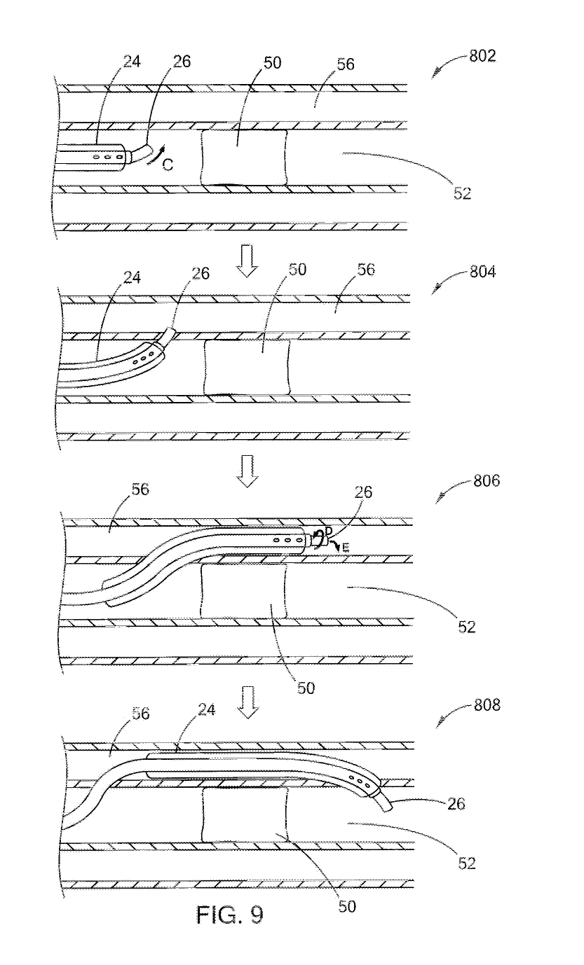

FIG. 9 shows steps of one method of treating an occlusion 50 in a body vessel having a true lumen 52 and a subintimal space 56. The occlusion may have a first side 12 and a second side 14, as illustrated in FIG. 1. In this example, the physician positions the wire guide into the true lumen 52 adjacent to the occlusion 50. The physician may sense the occlusion 50 based on a known method, such as tactile feedback or a visualization method. The physician advances the distal end of the elongate member along the wire guide adjacent to the first side of the occlusion 50.

In step 802, the physician advances the device from the first side of the occlusion 50 into the subintimal space 56, the cutting edge 26 being in the extended state. Through handle rotation and the tension mechanism, the physician positions the cutting edge 26 to cut into the subintimal space 56. This positioning may involve bending the cutting edge 26 in the direction of arrow C. In one aspect of the method, it is understood that the device may naturally line up with the intimal layer, such that there will be no need to rotate or bend the cutting edge to enter into the subintimal space 56. The cutting edge may naturally cut into the subintimal space based on its position. However, in most cases the user may need to orient the device to penetrate into the subintimal layer.

In step 804, the physician has cut into the subintimal space 56. After penetration, the physician may retract the cutting edge 26 into the retracted state and move past the occlusion 50. In step 806, the device is advanced past the occlusion 50. The physician now may desire to re-enter the true lumen 52. In this case, the user may advance the distal end from the subintimal space 56 into the true lumen 52 and adjacent to the second side of the occlusion 50 having the cutting edge being in the extended state. Again, this may involve extending the cutting edge into the extended position, rotating the device and the cutting edge 26 about the circumference in the direction of arrow D, and applying a tension on the cutting edge 26 in the direction of arrow E by way of the tension mechanism to move the cutting edge 26 between the straight state and the bent state. This same action occurs regardless of if the cutting edge is biased to be bent or straight. The physician may make the cut through the intimal layer into the true lumen 52. At this point, the physician may retract the cutting edge 26 into the retracted state after the step of advancing.

In step 808, when the device may be moved through the body vessel and advanced into the true lumen 52, the user may retract the cutting edge 26 and align the expandable balloon 24 adjacent to the occlusion 50. In one form, the expandable balloon 24 is maintained within the subintimal space 56. The user may expand the expandable balloon from the collapsed state to the expanded state to reduce or ablate the occlusion 50 against the vessel wall. Once the occlusion is pushed to the side, the user may deflate the expandable balloon, and retract the elongate member and the wire guide from the body vessel. At this point, the user has cleared the vessel's true lumen. This may allow blood flow to resume through its natural pathway.

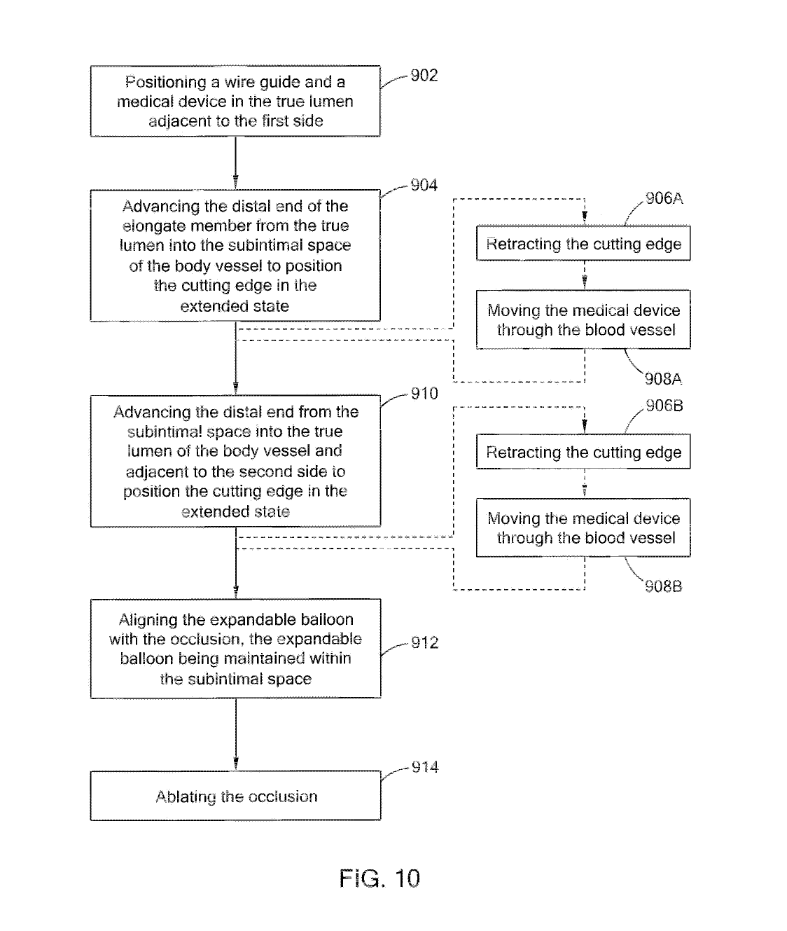

FIG. 10 shows a flow diagram of the treatment. In step 902, the physician positions a wire guide and a medical device in the true lumen adjacent to the first side of the occlusion. In step 904, the physician advances the distal end of the elongate member from the true lumen into the subintimal space of the body vessel having the cutting edge in the extended state. As needed, the physician may rotate the device about the circumference while performing the steps of advancing the distal end. This rotation may optimally position the cutting edge to make a cut. Likewise, when the cutting edge is extended, the physician may apply a tension to the cutting edge as needed to move the cutting edge between the straight and bent states, before the step of retracting the cutting edge to the retracted state.

In step 906A, the physician may retract the cutting edge to the retracted state for a first time, after the step of advancing the distal end. Likewise in step 906B, the physician may move the medical device through the vessel.

In step 910, the physician advances the distal end from the subintimal space into the true lumen of the body vessel and adjacent to the second side having the cutting edge in the extended state. Again, the physician may go through steps 906B and 908B, respectively. In step 908A, the physician may retract the cutting edge to the retracted state for a second time. In step 908B, the physician may move the device through the vessel. In step 912, the physician aligns the expandable balloon with the occlusion, the expandable balloon being maintained within the subintimal space. In step 914, the user will ablate the occlusion, by inflating and deflating the expandable balloon. At this point the occlusion has been pushed against the vessel wall and the vessel will be open for blood flow. Finally, the user withdraws the elongate member and the wire guide from the vessel.

FIGS. 11A and 11B depict a delivery assembly 200 for introducing and retrieving the device. As shown, the delivery assembly 200 includes a polytetrafluoroethylene (“PTFE”) introducer sheath 202 for percutaneously introducing an outer sheath 204 into a body vessel. Of course, any other suitable material for the introducer sheath 202 may be used without falling beyond the scope or spirit of the present invention. The introducer sheath 202 may have any suitable size, for example, between about 3-FR to 8-FR. The introducer sheath 202 serves to allow the outer sheath 204 and the elongate member or catheter 220 to be percutaneously inserted to a desired location in the body vessel. The inner member may also include, for example, a stylet. The introducer sheath 202 receives the outer sheath 204 and provides stability to the outer sheath 204 at a desired location of the body vessel. For example, the introducer sheath 202 is held stationary within a common visceral artery, and adds stability to the outer sheath 204, as the outer sheath 204 is advanced through the introducer sheath 202 to a filter area in the vasculature. The outer sheath 204 has a body extending from a proximal end 216 to a distal end 210, the body being tubular and including a sheath lumen extending therethrough.

As shown, the assembly 200 may also include a wire guide 208 configured to be percutaneously inserted within the vasculature to guide the outer sheath 204 to the occlusion. The wire guide 208 provides the outer sheath 204 with a path to follow as it is advanced within the body vessel. The size of the wire guide 208 is based on the inside diameter of the outer sheath 204 and the diameter of the target body vessel.

A needle may also be used. The needle may be used for percutaneously introducing the wire guide into the patient's body through an access site. A cutting device may also be used to expand the access site.

The elongate member 220 extends from a proximal portion 211 to a distal portion 212 and is configured for axial movement relative to the outer sheath 204 via the handle 240. Handle 240 may be integrally formed with the elongate member 220 or it may be attached to the elongate member 220 by any method known in the art. This may include gluing, bonding, welding, and the like. In this example, the distal portion 212 is shown adjacent to the distal end 218.

The outer sheath 204 further has a proximal end 216 and a hub 218 to receive the elongate member 220 advanced therethrough. The size of the outer sheath 204 is based on the size of the body vessel in which it percutaneously inserts, and the size of the elongate member 220. In this embodiment, the elongate member 220 is coaxially advanced through the outer sheath 204. In order to more easily deploy the elongate member 220 into the body vessel, it may have a lubricious coating, such as silicone or a hydrophilic polymer, e.g. AQ® Hydrophilic Coating as known in the art. Likewise, the elongate member 220 may be retracted through the outer sheath 204.

It is understood that the assembly described above is merely one example of an assembly that may be used to deploy the device in a body vessel. Of course, other apparatus, assemblies and systems may be used to deploy any embodiment of the device without falling beyond the scope or spirit of the present invention.

While the present invention has been described in terms of certain preferred embodiments it will be understood that the invention is not limited to this disclosed embodiments as those having skill in the art may make various modifications without departing from the scope of the following claims.

1. A medical device for treating an occlusion in a body vessel, comprising:
an elongate member having a proximal end extending distally to a distal end, the elongate member having a circumference and a plurality of lumens formed therethrough, the plurality of lumens comprising a cutting lumen, a balloon inflation lumen, and a wire guide lumen;
a cutting edge slidably disposed within the cutting lumen and having an arcuate cross-section extending up to half of the circumference, the cutting edge comprising a first end extending distally to a second end, the cutting lumen being formed complementary to the cutting edge;
an extending member operable to move the cutting edge relative to the cutting lumen, defining an extended state and a retracted state of the cutting edge;
a tension mechanism being connected to the proximal end and extending to the cutting edge, the tension mechanism moveable in a predetermined direction to bend the cutting edge, defining a straight state and a bent state of the cutting edge; and
an expandable balloon disposed circumferentially about the elongate member and in fluid communication with the balloon inflation lumen wherein the expandable balloon moves from a collapsed state to an expanded state to treat the occlusion.

2. The medical device of claim 1 wherein the elongate member comprises a plurality of side ports disposed adjacent to the distal end to provide fluid communication between the balloon inflation lumen and the expandable balloon.

3. The medical device of claim 1 wherein the proximal end comprises a handle and the tension mechanism comprises a tension core, the tension core being connected to the handle and extending distally to the cutting edge to move the cutting edge between the straight and bent states by applying a tension to the cutting edge.

4. The medical device of claim 3 wherein the tension core comprises a wire.

5. The medical device of claim 1 wherein the elongate member comprises a radiopaque marker at the distal end, the radiopaque marker being disposed adjacent to the cutting lumen.

6. The medical device of claim 5 wherein the radiopaque marker is L-shaped for positioning the device.

7. The medical device of claim 6 wherein the handle is integrally formed with the elongate member at the proximal end.

8. The medical device of claim 7 wherein the handle comprises a tension lever being depressible and releasable to move the cutting edge between the straight and bent states.

9. The medical device of claim 1 wherein the cutting edge extends from the proximal end to the distal end.

10. The medical device of claim 1 wherein the elongate member forms a track closer to the proximal end than the distal end, the extending member comprising a button being slidably received in the track.

11. The medical device of claim 1 further comprising a push member being connected to the proximal end and extending distally to the first end distal the proximal end.

12. The medical device of claim 1 wherein the expandable balloon is disposed proximal to the distal end.

13. The medical device of claim 1 wherein the cutting edge comprises a flexible material.

14. The medical device of claim 12 wherein the flexible material comprises shape memory material.

15. A method for treating an occlusion in a body vessel having a true lumen and a subintimal space, the occlusion having a first side and a second side, the method comprising:
positioning a wire guide and a medical device in the true lumen adjacent to the first side, the medical device comprising:
an elongate member having a proximal end extending distally to a distal end, the elongate member having a circumference and a plurality of lumens formed therethrough, the plurality of lumens comprising a cutting lumen, a balloon inflation lumen, and a wire guide lumen;
a cutting edge slidably disposed within the cutting lumen and having an arcuate cross-section extending, the cutting edge comprising a first end extending distally to a second end, the cutting lumen being formed complementary to the cutting edge;
an extending member operable to move the cutting edge relative to the cutting lumen, defining an extended state and a retracted state of the cutting edge;
a tension mechanism being connected to the proximal end and extending to the cutting edge, the tension mechanism moveable in a predetermined direction, defining a straight state and a bent state of the cutting edge; and
an expandable balloon disposed circumferentially about the elongate member and in fluid communication with the balloon inflation lumen wherein the expandable balloon moves from a collapsed state to an expanded state to treat the occlusion;
advancing the distal end of the elongate member from the true lumen into the subintimal space of the body vessel having the cutting edge being in the extended state;
advancing the distal end from the subintimal space into the true lumen of the body vessel adjacent to the second side having the cutting edge being in the extended state; and
aligning the expandable balloon with the occlusion, the expandable balloon being maintained within the subintimal space.

16. The method of claim 15 wherein the steps of advancing the distal end from the true lumen and advancing the distal end from the subintimal space both comprise rotating the device about the circumference.

17. The method of claim 15 wherein the steps of advancing the distal end from the true lumen and advancing the distal end from the subintimal space both comprise applying a tension on the cutting edge to move the cutting edge between the straight and bent states.

18. The method of claim 15 further comprising retracting the cutting edge to the retracted state for a first time after the step of advancing the distal end from the true lumen and before the step of advancing the distal end from the subintimal space.

19. The method of claim 18 further comprising retracting the cutting edge to the retracted state for a second time after the step of advancing the distal end from the subintimal space and before the step of aligning the expandable balloon.

20. The method of claim 19 further comprising moving the distal end through the body vessel after the step of retracting the cutting edge for the first time and before the step of advancing the distal end from the subintimal space.
